(19) United States
(12) Patent Application Publication
(10) Pub. No.: US 2025/0275565 A1
(43) Pub. Date: Sep. 4, 2025

(54) TERPENE-REDUCED CANNABINOID ADJUNCT

(71) Applicant: JAMAICAN BREW HOUSE, Grand Cayman (KY)

(72) Inventors: Jaime José JURADO, Forty Fort, PA (US); Erin WINCZURA, George Town (KY)

(21) Appl. No.: 18/920,201

(22) Filed: Oct. 18, 2024

Related U.S. Application Data
(63) Continuation of application No. 17/067,088, filed on Oct. 9, 2020, now abandoned.
(60) Provisional application No. 62/913,502, filed on Oct. 10, 2019.

(51) Int. Cl. A23L 33/105; A23B 2/40; A23L 5/20; A23L 29/30; A23L 33/14; A23L 33/185; A23L 33/21

(52) U.S. Cl. CPC A23L 33/105; A23B 2/40; A23L 5/273; A23L 29/30; A23L 33/14; A23L 33/185; A23L 33/21; A23V 2002/00

(57) ABSTRACT

Herein are described processes for the preparation of a cannabinoid adjunct. The process includes contacting a first plant matter having a first cannabinoid to terpene (C/T) ratio with a solvent at a temperature below about 0° C. for at least 1 hour producing a first solvent fraction. The first solvent fraction is separated from the first plant matter and fractionated providing a second solvent fraction, a terpene fraction and a solvent-free cannabinoid fraction. The cannabinoid fraction has a second C/T ratio that is greater than or equal to the first C/T ratio. The cannabinoid fraction can be combined with a soluble fiber thereby producing the cannabinoid adjunct. The cannabinoid adjunct is suitable for use in edible products.

### PRIORITY CLAIM

This application is a continuation of U.S. patent application Ser. No. 17/067,088 filed Oct. 9, 2020, which claims priority under 35 U.S.C. § 119 to U.S. Provisional Patent Application No. 62/913,502 filed Oct. 10, 2019, the contents of each of which are incorporated herein by reference in their entirety.

### FIELD OF THE INVENTION

This disclosure relates to a cannabinoid adjunct, its preparation and uses thereof. More particularly, the disclosure relates to a cannabinoid adjunct having a reduced terpene content, where the cannabinoid can be used as an ingredient in an edible product.

### BACKGROUND

The cannabis plant is largely known for yielding cannabinoids, especially tetrahydrocannabinol (THC) and cannabidiol (CBD), although the plant also biosynthesizes other cannabinoids. Where THC is known as a psychoactive compound, CBD is sometimes considered non-psychoactive but purportedly can be used for insomnia, anxiety, pain and generally wellness. The FDA has recently approved a plant based purified, crystalline, form of CBD EPODIOLEX® as a treatment for Lennox-Gastaut syndrome or Dravet syndrome in patients 2 years of age and older. Where an isolated crystalline cannabinoid is often desirable in a pharmaceutical grade drug, such a single component chemical in an edible material is generally not desired. For example, pure ethanol is not as desirable to ingest as a wine, beer or whisky. In addition to general taste, any synergistic effects of a combination of cannabinoids would be missing. The combination of cannabinoids with edible ingredients can also be challenging due to cannabinoids lipophilic character and low solubility in water.

In addition to cannabinoids, the cannabis plant contains over 100 varieties of terpenes. This is a large and diverse group of volatile oils that are found in numerous plants and are what give cannabis their characteristic scent and flavor. Although terpenes provide the signature smells and flavors to the specific variety of cannabis, and purportedly provide other benefits, the control of these without any processing is challenging. For example, the various terpene profiles can depend on not only the cannabis plant variety but also on the growing conditions such as nutrients available, water available and even other environmental factors such as the flora and fauna. In addition, some terpenes can impart a pleasant taste or smell if inhaled but may have a bitter taste if ingested.

Therefore, there is a need for a plant based cannabinoid adjunct that is low in terpenes, includes at least one cannabinoid and is suitable as a food additive.

### SUMMARY

In general, the description herein relates to a cannabinoid adjunct and edible products made incorporating the cannabinoid adjunct into the edible products. The processes described herein for making the cannabinoid adjunct provide an adjunct have a high cannabinoid to terpene (C/T) ratio from a plant matter.

In one aspect, the disclosure is for a process for the preparation of a cannabinoid adjunct, the process comprising: (a) contacting a first plant matter having a first cannabinoid to terpene (C/T) ratio with a solvent at a temperature below about 0° C. for at least 1 hour producing a first solvent fraction; (b) separating the first solvent fraction from the first plant matter; (c) fractionating the first solvent fraction producing a second solvent fraction, a terpene fraction and a solvent-free cannabinoid fraction, wherein the cannabinoid fraction has a second C/T ratio that is greater than or equal to the first C/T ratio; (d) combining the cannabinoid fraction with a soluble fiber thereby producing the cannabinoid adjunct.

In a second aspect, the disclosure is for a terpene-reduced cannabinoid adjunct made according to the first aspect. The terpene-reduced cannabinoid adjunct comprising at least one of tetrahydrocannabinol (THC), cannabidiol (CBD), cannabichromene (CBC), canabinol (CBN), cannabielsoin (CBE), cannnabicyclol (CBL), cannabicitran (CBT), or isomers thereof.

In a third aspect, the disclosure is for an edible product comprising the terpene-reduced cannabinoid according to the second aspect. Optionally, the cannabinoid adjunct is homogeneously distributed throughout the edible product.

In a fourth aspect, the disclosure is for a method of making an edible product. The method comprising combining a cannabinoid adjunct made according to the first aspect with water and at least one edible ingredient, thereby providing an edible combination.

The processes and products as disclosed herein provide a plant derived cannabinoid adjunct having a high C/T ratio and high mixing or dispensability with other edible ingredients. This provides a naturally derived food adjunct readily used for making edible products with one or more cannabinoids.

The above summary is not intended to represent every embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims

### BRIEF DESCRIPTION OF THE FIGURES

The disclosure will be better understood from the following description of exemplary embodiments together with reference to the accompanying drawings.

FIG. 1 shows a process for preparation of a cannabinoid adjunct from a cannabinoid containing plant matter, according to some implementations.

FIG. 2 illustrates steps for preparing a liquid high-purity adjunct, according to a first implementation.

FIG. 3 illustrates steps for preparing a liquid high-purity adjunct, according to a second implementation.

FIG. 4 illustrates steps for preparing a liquid high-purity adjunct, according to a third implementation.

FIG. 5 illustrates steps for producing a fermented beverage using a cannabinoid adjunct, according to some implementations.

The present disclosure is susceptible to various modifications and alternative forms. Some representative embodiments have been shown by way of example in the drawings and will be described in detail herein. It should be understood, however, that the inventions are not intended to be limited to the particular forms disclosed. Rather, the disclosure is to cover all modifications, equivalents, and alternatives falling within the spirit and scope of the inventions as defined by the appended claims.

### DETAILED DESCRIPTION

The present disclosure relates to processes for preparation of a terpene-reduced cannabinoid adjunct extracted for a plant. The disclosure also is for terpene-reduced cannabinoid adjunct and its uses. For example, the terpene-reduced cannabinoid adjunct is suitable as a neutral tasting ingredient used for beverages and other edibles.

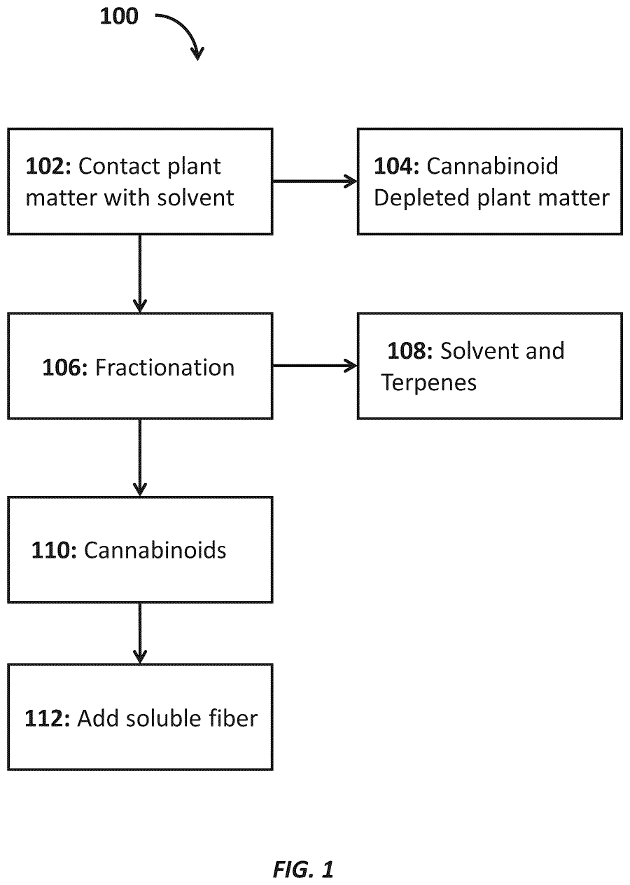

FIG. 1 shows a process 100 for preparation of a cannabinoid adjunct from a cannabinoid containing plant matter. The process provides an enhancement of the C/T ratio as compared to the initial plant matter. Therefore, prior to application of process 100 the C/T ratio from the plant is lower than the C/T ratio of at least one product derived from application of process 100. In a first step 102, a first plant matter is contacted with a solvent. This contact extracts at least of portion of the cannabinoids and terpenes from the plant matter, providing a cannabinoid and terpene enrichment in a first solvent fraction and a cannabinoid and terpene depletion in the plant matter. The cannabinoid depleted plant matter is separated from the cannabinoid and terpene rich first solvent in a second step 104. The first solvent, enriched in cannabinoids and terpenes, is then subjected to a fractionation 106. Fractionation 106 separates the solvent and terpenes 108 from the cannabinoids 110. The cannabinoids 110 are combined with a soluble fiber providing a terpene free cannabinoid adjunct 112 suitable for further processing, such as for use in making edible products. In some implementations, the process 100 can include further steps, before or after, any one of steps 102, 104, 106, 108 or 110. In some other implementations, the process can exclude one or more of the steps, for example addition of a soluble fiber 112 can be excluded, or the fractionation step 106 can be excluded.

In some implementations, the first plant matter, prior to being contacted with the solvent 102, is subjected to drying. In some implementations, the drying is in ambient air. In some implementations the first plant matter is subjected to a drying environment for at least one day, wherein the dry environment has a relative humidity less than about 90% and a temperature in a range of 15-40° C. In some implementations, the first plant matter is placed in the dry environment for at least 1, 2, 3, 4, 5, 6 or 7 days and any range bounded by two of these values. In some implementations the humidity is less than about 85, 80, 75, 70, 65, 60, 55, 50, 40, 45, 40, 35, 30, 25, 20, or 15, and any range bounded by two of these values. As used herein, the humidity is the relative humidity. In some implementations the temperature is about 15, 16, 17, 18, 19, 20, 21, 22, 23, 24, 25, 26, 27, 28, 29, 30, 31, 32, 33, 34, 35, 36, 37, 38, 39 or 40° C., and any range bounded by two of these values. In some implementations, the first plant matter is placed in the dry environment for 1-7 days, and wherein the humidity is less than about 85% and the temperature is in a range of 19-35° C.

In some implementations, the drying provides an initial enhancement to the C/T ratio, such as by allowing some of the terpenes to be removed into the ambient air of the drying environment. Without being bound by any specific theory, it is also suggested that this drying provides a preliminary oxidation of the plant material.

In some implementations, prior to contacting the first plant matter to solvent 102 the first plant matter is subjected to a first trim. For example, wherein the first trim comprises removal of stems and leaves from the first plant matter. In some implementations, the “fan” or large leaves connected to the stems are removed. This provides a first plant matter that is enhanced in the flower or “Bud” of the plant where cannabinoid rich trichomes reside, and a first trim material.

In some implementations, after a first trim, the first plant matter is subjected to a second or fine trim. For example, to remove any seeds or smaller leaves and stems from the flowers. In some implementations the fine trim removes the “sugar” leaves, or small leaves, that are connected directly to the flower. In some implementations, stems are removed from the flower, such as any stems not removed in the first trim. This provides a flower substantially free of any leaves, wherein substantially means less than 5% mass (e.g., less than 1% mass) of the flower includes any leaves such as sugar leaves. This also provides a second trim material, such as including stems and leaves.

The first and second trim can be done by any useful method, both mechanical or by hand. For example, commercially available trimmers, such as the Munch Machine (Dauenhauer Manufacturing, Inc.) can aid in this process for removing the stems from the flowers after larger “fan leaves” are removed from the stem. Hand held electric trimmers, such as the Magic Trimmer (Trim Buddies LLC, CA) can be used for removal of leaves on the flower or “sugar” leaves. Other mechanical trimmers such as the CenturionPro trimmers (Centurion Pro Solutions, Canada), or TRIMINATOR® trimmers (Triminator, CA) can also be used.

After the first and second trim, where implemented, the flower remains as the first plant material to be contacted with the solvent 102. Depending on the variety of plant used, the amount of flowers account for between about 5% to about 30% of the plant material. For example, in some implementations using a cannabis strain common in Jamaica, “Jamaican”, which is an indigenous or landrace cannabis strain, the flowers typically account for about 15% of the plant.

In some implementations, the trim plant matter (e.g., the first trim and second trim), such as leaves and stock and not including the flower, is discarded. In some implementations, at least a portion of the trim plant or matter is repurposed. In some implementations, at least a portion of the trim material, such as fibrous portions, can be used as a fuel, building material, textile or a feed source. In some implementations, at least a portion of the trim plant material is further processes such as by a hammer mill, a pulper, an agricultural shredder, a press, a juicer, a blender or a combination thereof. In some implementations, the trim plant material is processed to produce a particulate, macerate or pulp material. In some implementations, at least a portion of the trim plant material, such as the fan leaves, is processed to provide a leaf protein. In some implementations, the leaf protein is used as a nutrient source for a brewing yeast.

In some implementations, prior to contacting the first plant matter to solvent 102, the first plant matter is subjected to a decarboxylation, wherein after the decarboxylation the first C/T ratio is greater than a C/T ratio of the first plant matter prior to decarboxylation. As used herein, decarboxylation refers to the transformation of the acid form of cannabinoids as they are biosynthesized and initially extracted from plants, and where a carbon dioxide molecule is removed, to yield the decarboxylated form. For example, the decarboxylation transforms tetrahydrocannabinolic acid (THCA) to tetrahydrocannabinol (THC), and cannabidiolic acid (CBDA) to cannabidiol CBD. The decarboxylation can be completed to any degree, such as so that at least 10%, 20%, 30%, 40%, 50%, 60%, 70%, 80%, 90%, 95%, 99% or even 100% of the starting cannabinoid (acid form) is decarboxylated.

In some implementations, the decarboxylation comprises heating the first plant matter at a temperature in a range of about 90-120° C. For example, the temperature during the decarboxylation can be 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120° C., and in any range bounded by two of these temperatures. In some implementations, the plant matter is set on tray, or moving belt (e.g., for a continuous process) and exposed to heat in any suitable oven for the decarboxylation. As used herein, “oven” refers to any suitable space that provides a heated environment of air wherein the first plant matter can be placed including a convection oven, a conventional oven (e.g., non-convection), rotary oven, an air fryer, and tunnel ovens. In some implementations, the oven can be under vacuum or under flowing air. In some implementations, the heating for the decarboxylation lasts at least 10 min. In some implementations, the heating is for about 10 to 120 min. For example, the heating can be for 10, 20, 30, 40, 50, 60, 70, 80, 90, 100, 110 or 120 minutes, and any range bounded by two of these times. In some implementations, the decarboxylation includes heating the first plant material at about 107° C. (225° F.) for about 25 min.

In some implementations, a combination of times and ranges of temperatures can be used, providing, for example, temperature ramps (positive and negatives) held temperatures and combinations of these. In some implementations, one or more decarboxylation steps can be used, including different temperatures, temperature ramps, and using different ovens.

In some implementations, the decarboxylation is done before the first trim. In some implementations, the decarboxylation is done after the first trim and before the second trim. In some implementations, the decarboxylation is done after the second trim.

In some implementations, the first plant matter is contacted with a solvent 102 where the solvent is ethanol, a hydrocarbon or supercritical CO₂. The solvent can be contacted with the plant matter in a continuous, semi-continuous or batch manner. For example, in implementations where a continuous process is used, a tube type reactor can be charged with the first plant matter and the solvent made to flow through the plant matter. Optionally, heating or chilling can be provided to the tube by external heaters or chillers. In implementations where the process is a batch process, the plant material can be placed in a container and the solvent added to the container. The container can be a pressurized container or a container under atmospheric pressure optionally including a cover. Optionally, the batch process includes contacting the material with the solvent for a set amount of time and then the solvent is separated from the biomass. Separation can be by any suitable means such as a filtration, decanting, using a centrifuge or any combination of these. Filtering can include using any filter having any suitable mesh size (e.g., 4-400) or distribution of mesh sizes. Optionally, the filter can include as a cheese cloth, a sieve, a filter bag, a filter paper, or a strainer. Optionally one or more filter or filtering method can be used. In some implementations, the filtering includes a vacuum or pressurized filtration, a gravity filtration or a centrifuge filtration.

In some implementations, the solvent is ethanol such as an ethanol having a 60%-90% abv. In some implementations, the solvent is contacted with the first plant matter at a temperature in a range of between about −40 to 0° C. For example, the temperature can be about −40, −35, −30, −25, −20, −15, −10, −5, or 0° C.; or selected in any range between two of these temperatures. In some implementations, the solvent is contacted with the first plant matter 102 for less than about 5 days, such as less than about 6 days, 4 days, 72 hr, 48 hr, 40 hr, 36 hr, 32 hr, 28 hr, 24 hr, 20 hr, 16 hr, 8 hr or 4 hr; or any time selected within a range bounded by two of these times. In some implementations, contacting the first plant matter with a solvent 102 is at a temperature in a range of about −40 to 0° C. for less than about 72 hours producing the first solvent fraction. In some implementations, contacting the first plant matter with a solvent 102 is at a temperature in a range of about −40 to 0° C. for less than about 48 hours producing the first solvent fraction. In some implementations, contacting the first plant matter with a solvent 102 is done at a temperature in a range of about −40 to 0° C. for less than about 48 hours producing the first solvent fraction. In some implementations, the ethanol is contacted with the first plant material in a batch process. The solvent can be contacted with ethanol using commercially available extraction equipment such CUP-15 and CUP-30 extractors (Delta Separation, CA), and ETHOS® extractors (Capna Labs, CA).

In some implementations, during contacting the plant matter to solvent, a weight range of one part plant matter to 10 parts solvent (1:10) up to one part cannabis to 30 parts solvent (1:30) is used. For example, the ratio of solvent to plant matter can be; 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29 or 1:30; or any range between and including two of these ratios.

In some implementations, a hydrocarbon extraction is used. For example, a propane, a butane, or hexane extraction can be used. Some commercial extractor includes closed loop propane and butane systems (ExtractionTek Solutions, CO).

In some implementations, a carbon dioxide extraction can be used. For example, super critical CO₂ extractors are commercially available (e.g., Medxtractor Corp., Canada; Vitalis Extraction Technologies, Canada; Eden Labs LLC, WA)

In some implementations, the depleted plant matter is discarded. In some implementations, the depleted plant mater is re-purposed or re-used. For example, the depleted plant matter can be used as fuel or ground and used a feed. In some implementations, the depleted plant matter can be pulped and used as a source of leaf protein.

In some implementations, fractionating the first solvent fraction 106 comprises fractional distillation. In some implementation, the fractional distillation comprises using a spin band column. As used herein, a spin band column is used for spinning band distillation which is a technique that uses a rotating helical band to create a high number of theoretical plates. In some implementations, the fractional distillation is conducted under a vacuum in a range of 0.5 to 50 torr. For example, 1, 5, 10, 15, 20, 25, 30, 35, 40, 45 or 50 torr; or any range bounded by these values. In some implementations, a short path distillation is used. In some implementations, a wiped film distillation is used. In some implementations, a reflux distillation is used. In some implementations, fractionating the first solvent fraction 106 comprises fractional distillation using a solvent recovery system such as a rotary evaporator, for example an ECODYST® Rotovap (Ecodyst, Inc., NC)

In some implementations, the cannabinoid fraction 110 is a liquid. In some implementations, the cannabinoid fraction is a liquid having a high viscosity at room temperature such as at least about 2000 mPas. For example, the viscosity can be at least 7000, 10,000 or even at least 20,000 mPas at room temperature. In some implementations, the cannabinoid fraction 110 includes at least one of tetrahydrocannabinol (THC), cannabidiol (CBD), cannabichromene (CBC), canabinol (CBN), cannabielsoin (CBE), cannnabicyclol (CBL), cannabicitran (CBT), or isomers thereof. In some implementations, the cannabinoid fraction 110 includes at least two cannabinoids. In some implementations, the cannabinoid fraction 110 includes at least three cannabinoids. In some implementations, the cannabinoid fraction 110 comprises at least about 50% THC, such as at least about 60%, 70%, 80%, 90%, 95% or 99% THC. In some implementations, the cannabinoid fraction 110 comprises at least about 50% CBD, such as at least about 60%, 70%, 80%, 90%, 95% or 99% CBD. In some implementations, the C/T ratio of the cannabinoid fraction 100 is at least 100, as at least 1000, or at least 10,000. In some implementations, the C/T ratio of the cannabinoid fraction 100 approaches infinity, where no terpenes can be detected by conventional methods such as gas chromatography or high pressure liquid chromatography. In some implementations, the cannabinoid fraction is further processes, for example, by recrystallization to provide a substantially pure cannabinoid in a crystalline form, e.g., >99%, THC or >99% CBD. In some implementations, the cannabinoid fraction 100 is considered a “broad spectrum” product, wherein a broad spectrum as used herein refers to a product having more than one cannabinoid, such as 2, 3, 4, 5 or more cannabinoids.

In some implementations, the cannabinoids 110 are combined with a soluble fiber or other material selected from xantham gum, guar gum, maltose, fructose, and maltotriose. In some implementations, the soluble fiber is xantham gum. As used herein, combined can include by one or more of mixing, sonicating, and blending. In some implementations, the combining process provides a homogeneous mixture.

As previously described, fractionation 106 separates the solvent and terpenes 108 from the cannabinoids 110. In some implementations, the solvent is repurposed such as to be used for fuel. In some implementation, the solvent is recycled and used again in process 100 for extraction. In some implementations, the solvent is processed, for example distilled a second time, prior to being re-purposed or recycled. In some implementations, the terpene fraction is discarded. In some implementations, the terpene fraction is used, for example, as an additive in an edible material. In some implementations, the terpene fraction is further processed or separated into different components, for example, by further distillation, crystallization, chromatography or any other suitable separation technique.

In some implementations, the process 100 according to FIG. 1, further comprises reducing the volume (e.g., concentrating) of the first solvent fraction by at least 30%, wherein after reducing the volume, the first C/T ratio is greater than a C/T ratio prior to reducing the volume. In some implementations, reducing the volume of the first solvent fraction comprises reducing the volume by at least about 60% or at least about 65%. In some implementations, reducing the volume comprises a distillation.

In some implementations, after reducing the volume of the first solvent fraction, the process further comprises cooling the first solvent fraction at a temperature below about 0° C. for at least 1 hour. For example, the temperature can be less than about −5, −10, −15, −20, −25, −30 or-35° C. In some implementations, the temperature during cooling is maintained for at least 1 hour, at least 2hours, at least 6 hours, at least 12 hours, or at least 24 hours. In some implementations, the temperature during cooling is maintained during cooling for less than about 7 days, such as less than about 6, 7, 5, 4, 3, 2 or 1 day. In some implementations, cooling the first solvent fraction is at a temperature in a range of about −40 to 0° C. for less than about 24 hours.

In some implementations, after cooling the first solvent fraction, the process further comprises separating a lipid containing fraction from the first solvent fraction. The separation can be by any useful method, for example by vacuum filtration, decanting, centrifuge or a combination of these. As referred to herein, the lipid containing fraction can include other materials, such as waxes, fats and lipids.

In some implementations, process 100 further comprises contacting the first plant matter with steam prior to contacting the first plant matter to the solvent 102, wherein after contacting with steam the first C/T ratio is greater than a C/T ratio of the first plant matter prior to contacting with steam. The first plant matter is contacted with steam by any suitable means. For example, the material can be placed in a bag or porous enclosure (e.g., cheese cloth) and suspended over a source of steam. This procedure removes at least a portion of the terpenes without removing cannabinoids from the plant matter. In some implementations, contacting with steam, is done using steam distillation apparatus. In some implementations, contacting with steam or steam distillation is done more than once.

Without being bound to a specific mechanism, it is found that additional processing, as described herein, such as one or more of a decarboxylation step, a first trim, second trim, contacting with steam, and removing a lipid fraction after concentrating and cooling, renders the fractionation step 106 more efficient. For example, the time used in the fractionation step can be reduced by more than a half, or to less than about a third of the time, as compared to where one or more of these steps is omitted. The steps can be added in process 100 in any logical order. For example, the first trim occurs prior to the second trim, and contacting with steam occurs after a first, or a first and second trim, since a wet (e.g., steamed) product can be more difficult to trim. In some implementations, the decarboxylation step is omitted but a decarboxylation still occurs such as during the fractionation step 106 where heating can occur (e.g., distillation). In some implementations, the decarboxylation step is implemented before a first trim. In some implementations, the decarboxylation step is implemented after the first trim and before a second trim. In some implementations, the decarboxylation step is implemented after a second trim step. In some implementations all of the steps described herein are included.

In some implementations, the first plant matter comprises a plant of the genus Cannabis. In some implementations. the plant is Cannabis sativa, Cannabis indica, Cannabis ruderalis, or a hybrid of one or more of these. In some implementations, plants referred to as HEMP plants can also be used, for example, the US Hemp Farming Act of 2018 defines HEMP as cannabis with less than 0.3% THC.

In some implementations, the first plant matter includes one or more terpene selected from 1-octanol, alpha-2-pinene, beta-2-pinene, myrcene, alpha-phellandrene, delta-3-carene, beta-phellandrene, R-limonene, cineol, cis-ocimene, gamma-terpinene, terpinolene, (−)-linalool, beta-fenchol, cis-sabinene hydrate, camphor, borneol, alpha-terpineol, cis-bergamotene, beta-caryophyllene, trans-bergamotene, alpha-guaiene, aromadendrene, alpha-humulene, trans-beta-farnesene, gamma-selinene, delta-guaiene, gamma-cadinene, eudesma-3,7(11)-diene, gamma-elemene, nerolidol, trans-beta-caryophyllene, beta-caryophyllene oxide, guaiol, gamma-eudesmol, beta-eudesmol, phytol, pulegone, bisabolene, and alpha-bisabolol. In some implementations, at least one of these terpenes is removed from the plant material by the process 100. For example, at least 2, 3, 4, 5, 6, 7, 8, 9, 10, 15, 20, 50, 100 terpenes are removed by process 100.

In some implementations, an edible product can be made and includes an adjunct made by process 100, such as a terpene-reduced cannabinoid adjunct including a soluble fiber. In some implementations, the edible product is a liquid. For example, in some implementations the product is a fermented product (such as a beer, mead, cider or wine), a juice, a tea, a coffee, a frappe, a milk based drink (e.g., including nut and soy milk) or a protein drink. In some implementations, the edible product is a semi-solid such as a protein gel. In some implementations, the protein gel is a gummy (e.g., gummy bears), a desert gel (e.g., JELL-O®), or a soft toffee. In some implementations, the edible product is a solid such as a hard candy, a hard toffee, a candy bar (e.g., chocolate), a baked good (e.g., a cake, a bread, a brownie, or a cookie). In some implementations, the terpene-reduced cannabinoid adjunct is homogeneously dispersed in the edible product.

Some implementations are for a method of making an edible product comprising combining a cannabinoid adjunct, such as a terpene-reduced cannabinoid adjunct, with water and at least one edible ingredient, thereby providing an edible combination. In some implementations, combining comprises homogenizing the cannabinoid adjunct throughout the edible combination. For example, homogenizing by mixing, kneading, grinding, infusing, sonicating, blending or a combination of these. In some implementations, combining comprises baking or chilling, or any other useful food processing step. In some implementations, the edible ingredient is selected from any one or more of a starch, a sweetener, a flavoring agent, a coloring agent, a protein, an amino acid, a vitamin, a mineral, a fruit, an ester, a nutritional supplement, a nutraceutical, a spice, a flour (e.g., wheat, rice, nut, barley, corn or other edible flour), a fiber, a food grade oil (e.g., olive oil, coconut oil, MCT oil, palm oil, canola oil, corn oil, linseed oil, fish oil), a food grade fat, or an alcohol. In some implementations, the protein is a gel or a gel forming agent. In some implementations, the edible ingredient is a sweetener. In some implementations, the sweetener is a complex or simple sugar. In some implementations, the method further comprises partitioning the edible product into containers. In some implementations, the method further comprises pasteurizing the edible product. In some implementations, a terpene, such as one or more of the terpenes extracted from the plant used in process 100 is added to the edible product.

In some implementations, of a method for making an edible product, combining comprises further adding a yeast and a plant protein, and fermenting at least a portion of the sugar. For example, in some implementations fermenting comprises adding the yeast at a rate of 1 million cells/ml perDegree Plato (° P) liquid and with an initial temperature in a range between 5 and 20° C.

Implementations of various aspects described herein can be defined as in any of the following numbered paragraphs:
1. A process for the preparation of a cannabinoid adjunct, the process comprising:
   a. contacting a first plant matter having a first cannabinoid to terpene (C/T) ratio with a solvent at a temperature below about 0° C. for at least 1 hour producing a first solvent fraction;
   b. separating the first solvent fraction from the first plant matter;
   c. fractionating the first solvent fraction producing a second solvent fraction, a terpene fraction and a solvent-free cannabinoid fraction, wherein the cannabinoid fraction has a second C/T ratio that is greater than or equal to the first C/T ratio; and
   d. combining the cannabinoid fraction with a soluble fiber thereby producing the cannabinoid adjunct.
2. The process according to paragraph 1, further comprising:
   e. reducing the volume of the first solvent fraction by at least 30%, wherein after reducing the volume, the first C/T ratio is greater than a C/T ratio prior to reducing the volume;
   f. cooling the first solvent fraction at a temperature below about 0° C. for at least 1 hour; and
   g. separating a lipid containing fraction from the first solvent fraction.
3. The process according to paragraph 2, wherein reducing the volume of the first solvent fraction (step e) comprises reducing the volume by at least about 60%.
4. The process according to paragraph 2 or 3, wherein cooling the first solvent fraction (step f) is at a temperature in a range of about −40 to 0° C. for less than about 24 hours.
5. The process according to any one of paragraphs 1-4, further comprising contacting the first plant matter with steam prior to contacting the first plant matter to the solvent (step a), wherein after contacting with steam the first C/T ratio is greater than a C/T ratio of the first plant matter prior to contacting with steam.
6. The process according to any one of paragraphs 1-5, wherein prior to contacting the first plant matter to solvent (step a), the first plant matter is subjected to drying.
7. The process according to paragraph 6, wherein drying comprises placing the first plant matter in a dry environment for at least one day, wherein the dry environment has a relative humidity less than about 90% and a temperature in a range of 15-40° C.
8. The process according to paragraph 7, wherein the first plant matter is placed in the dry environment for 1-7 days, and wherein the humidity is less than about 85% and the temperature is in a range of 19-35° C.
9. The process according to any one of paragraphs 1-8, wherein prior to contacting the first plant matter to a solvent (step a), the first plant matter is subjected to a first trim, wherein the first trim comprises removal of stems and leaves from the first plant matter.
10. The process according to any one of paragraphs 1-9, wherein prior to contacting the first plant matter to solvent (step a), the first plant matter is subjected to a decarboxylation, wherein after the decarboxylation the first C/T ratio is greater than a C/T ratio of the first plant matter prior to decarboxylation.
11. The process according to paragraph 10, wherein the decarboxylation comprises heating the first plant matter at a temperature in a range of about 90-120° C. for at least 10 min.
12. The process according to any one of paragraphs 1-11, wherein prior to contacting the first plant matter to solvent (step a), the first plant matter is subjected to fine trim comprising isolating a flower from other plant components (e.g., stem and seeds) and using only the flower as the first plant matter which is contacted with the solvent.
13. The process according to any one of paragraphs 1-12, wherein the solvent is ethanol, a hydrocarbon or supercritical CO₂.
14. The process according to paragraph 13, wherein the solvent is ethanol having a concentration in a range of about 60%-90% abv.
15. The process according to any one of paragraphs 1-14, wherein contacting the first plant matter with a solvent (step a) is at a temperature in a range of about −40 to 0° C. for less than about 5 days producing the first solvent fraction.
16. The process according to any one of paragraphs 1-15, wherein fractionating the first solvent fraction (step c) comprises fractional distillation.
17. The process according to paragraph 16, wherein the fractional distillation comprises using a spin band column.
18. The process according to paragraph 16 or 17, wherein the fractional distillation is conducted under a vacuum in a range of 0.5 to 50 torr.
19. The process according to any one of paragraphs 1-18, wherein the first plant matter comprises a plant of the genus Cannabis.
20. The process according to paragraph 19, wherein the plant is Cannabis sativa, Cannabis indica, Cannabis ruderalis, or hydrides of one or more of these.
21. The process according to any one of paragraphs 1-21, wherein the soluble fiber (step d) comprises any one or more of xantham gum, guar gum, maltose, fructose, and maltotriose.
22. A terpene-reduced cannabinoid adjunct made according to any one of the preceding number paragraphs, comprising at least one of tetrahydrocannabinol (THC), cannabidiol (CBD), cannabichromene (CBC), canabinol (CBN), cannabielsoin (CBE), cannnabicyclol (CBL), cannabicitran (CBT), or isomers thereof.
23. An edible product comprising the cannabinoid adjunct according to paragraph 22.
24. The edible product according to paragraph 23, wherein the product is a solid, semisolid or liquid.
25. The edible product according to paragraphs 23 or 24, wherein the product is a fermented product.
26. The edible product according to any one of paragraphs 23-25, wherein the cannabinoid adjunct is homogeneously distributed throughout the edible product.
27. A method of making an edible product comprising;
   combining a cannabinoid adjunct made according to any one of paragraphs 1-21 with water and at least one edible ingredient, thereby providing an edible combination.
28. The method according to paragraph 27, wherein combining comprises homogenizing the cannabinoid adjunct throughout the edible combination.
29. The method according to any one of paragraphs 27-28, wherein the edible ingredient is selected from any one or more of a starch, a sweetener, a flavoring agent, a coloring agent, a protein, an amino acid, a vitamin, a mineral, a fruit, an ester, a nutritional supplement, a nutraceutical, a spice, fiber, a food grade oil, a food grade fat, or an alcohol.
30. The method according to paragraph 29, wherein the protein is a gel.
31. The method according to paragraph 29 or 30, wherein at least one edible ingredient is a sweetener.
32. The method according to paragraph 31, wherein the sweetener is a complex or simple sugar.
33. The method according to paragraph 32, wherein combining comprises further adding a yeast and a plant protein, and fermenting at least a portion of the sugar.
34. The method according to paragraph 33, wherein fermenting comprises adding the yeast at a rate of 1 million cells/ml perDegree Plato (° P) liquid and with an initial temperature in a range between 5 and 20° C.
35. The method according to any one of paragraphs 27-34, further comprising partitioning the edible product into containers.
36. The method according to any one of paragraphs 27-35, further comprising pasteurizing the edible product.

The embodiments will be more readily understood by reference to the following examples, which are included merely for purposes of illustration of certain aspects and embodiments of the present invention, and should not be construed as limiting. As such, it will be readily apparent that any of the disclosed specific constructs and experimental plan can be substituted within the scope of the present disclosure.

### EXAMPLES

### Example 1—Cannabinoid Adjunct Preparation 1

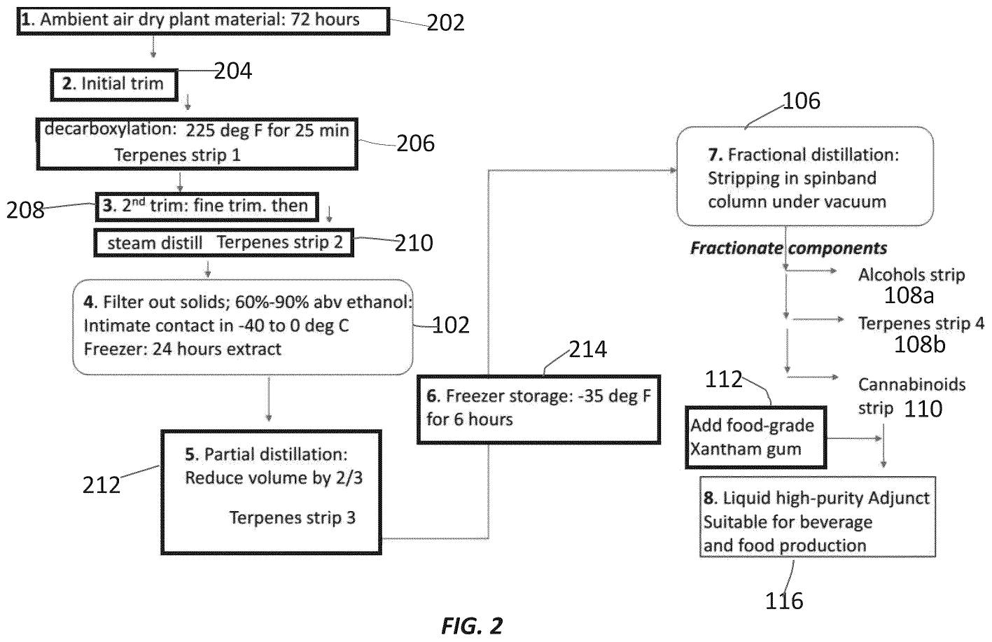

FIG. 2 illustrates steps for preparing a liquid high-purity adjunct suitable for beverage production. In a first step, 202 a harvested cannabis plant (Jamaican Strain landrace varietal: 13.5% THC estimate from literature) is dried in ambient air at a temperature between 19-35° C. for 3 days. An initial trim 204 removes the fan leaves and stalks. The flowers (Buds) are placed on a tray and the tray and flowers are heated in an oven at 225° C. (about 107° C.) for 25 min for decarboxylation 206. The decarboxylation 206 also includes a first terpene strip. A second or fine trim 208 follows decarboxylation 206. The fine trim 208 removes additional matter such as sugar leaves, seeds and any stems still attached to the flowers.

Following the fine trim, a steam distillation step is done 210. For the steam distillation, the flowers are placed in a cheese cloth and suspended over a kettle set to boil. The flowers are thereby exposed to the steam from the kettle. The process is repeated several times for an approximate exposure time of 45 min to steam. This procedure provides a second terpene strip from the flowers.

Following the steam distillation step 210, the flowers and ethanol (60% to 90% abv) are place in a container 102, where the ratio of ethanol to flowers is in a range of 1:10 to 1:30. The container is closed and chilled so that the temperature of the ethanol is in a range of 0 to −40° C. for 24 hours. After this treatment the material is filtered from the flower material to provide an ethanol solution containing cannabinoids. The ethanol is then reduced by ⅔ by a partial distillation 212. The partial distillation can be done using a rotary evaporator to reduce the solvent to about 10 wt % of the total mass, or a fractional distillation is used to reduce the solvent content to about 10 wt % of the total mass. In addition to concentrating the solvent, this partial distillation 212 provides a third terpene strip. Following this treatment, the ethanol containing cannabinoids is chilled at about −35° C. for 6 hours 214. The material can then be filtered to remove plant lipids/waxes and fats that precipitate out of solution.

Subsequent to these steps, the ethanol solution is subjected to a fractional distillation using a spinband column under vacuum (0.5 to 50 torr) in a fractionation step 106. Three fractions are collected. The fractionation 106 provides a solvent (alcohol) fraction 108a, a terpene fraction 108b and a cannabinoid fraction 110. The terpene fraction 108b provides a fourth terpene strip. The cannabinoid fraction 110 is yielded as a viscous solution with an amber color. The cannabinoid fraction is combined with xantham gum to provide the liquid high-purity cannabinoid adjunct 116.

### Example 2—Cannabinoid Adjunct Preparation 2

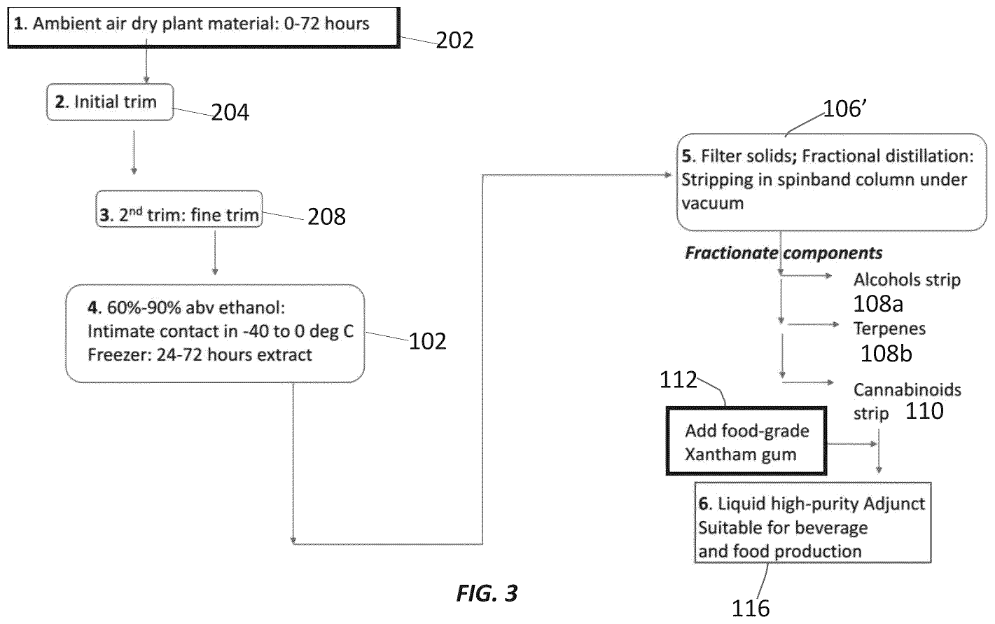

FIG. 3 illustrates an alternative series of steps for preparing a liquid high-purity adjunct suitable for beverage production. The steps are similar to those shown in FIG. 2 (Example 1) except that some steps are not included and some others are modified. In particular, the decarboxylation step 206, the steam distillation step 102, the partial distillation step 212, and the chilling step 214 are not included. A decarboxylation occurs during the fractionation step 106′. In addition, a filtering step is included prior to the fractional distillation and is included in fractionation step 106′. This second method also provide the liquid high-purity cannabinoid adjunct 116. Although this has fewer steps, most of the terpenes are removed only by the fractionation step 106′ which can make this step very slow. In addition, the chilling step 214 is eliminated so that any removal of lipids/fats and waxes is less efficiently removed in the filtration step combined with the fractionation step 106′.

### Example 3—Cannabinoid Adjunct Preparation 3

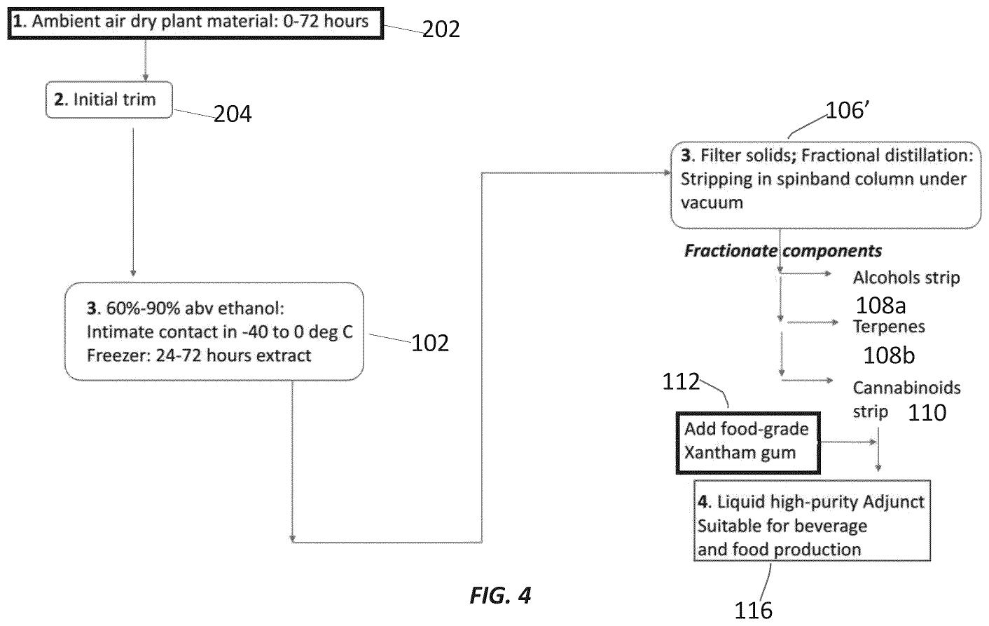

FIG. 4 illustrates another series of steps for preparing a liquid high-purity adjunct suitable for beverage production. The steps are similar to those described in Example 2 except that no second or fine trim 208 is done. This second method also provides the liquid high-purity cannabinoid adjunct 116. Although this has fewer steps, most of the terpenes are removed only by the fractionation step 106′, making this step slow. In addition, since the fine trim 208 is eliminated, the extraction step 102 can at times be less efficient since there is more plant material.

### Example 4—Preparation of a Fermented Beverage

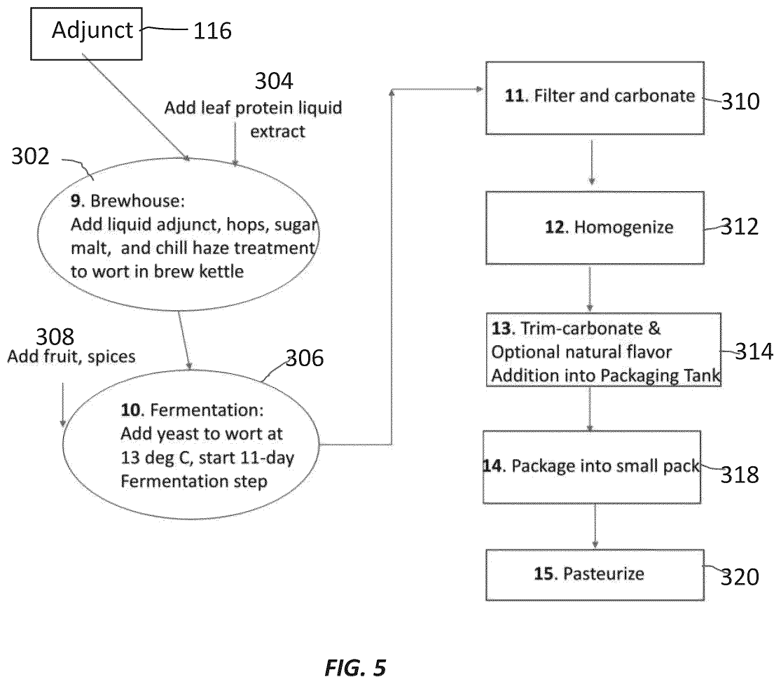

FIG. 5 illustrates steps for producing a fermented beverage using the adjunct 116. In an initial step the cannabinoid adjunct as prepared as described in examples 1, 2 or 3 can be combined with water, hops, sugar or malt extract 302. A leaf protein 304 can also be added. During the mashing process a brew kettle or Lauter Tun can be used and the material can be heated (e.g., boiled) as needed. A chiller can be used for a chill haze treatment, after which a brewer's yeast is added and the mixture is fermented 306 for 11 days. The fermentation rate is 1 million cells/ml per deg Plato liquid at an initial temperature of 13° C. Optionally fruits and spices or other food additive can be added during the fermentation 308.

Once the fermentation is complete, the fermented liquids are filtered or centrifuged, and carbonated 310. The liquids can be homogenized 312 if needed. A trim carbonation, optional natural flavor addition and transfer to a packing tank follows in step 314. The material is packaged into small containers 318 such as cans, bottles, kegs or barrels. A pasteurization step 320, Tunnel Pasteurization at 10-20 Pasteurization Units (PUs), completes the process.

### Experimental Data

A beer was made using the conditions of experiment 1 and experiment 4. The following data was collected:
14 g starting material: dried cannabis buds (Jamaican Strain landrace varietal: 13.5% THC estimate from literature)
12.4 g of plant material after initial trim.
11.7 g of plant material after decarboxylation.
10 g of plant material after fine trimming.
11.7 g plant material after wet-steam stripping.
300 ml alcohol contact below 0 deg C for 24 hours.
Filter out solids using nr. 4 paper filtration.
240 ml of liquid collected.
83 ml after distillation.
Simulate fractional distillation: reduce volume to 30 ml viscous fraction.
Add to 24 L ‘brews’ at brew kettle (boiling) phase and ferment in wort/liquid fermentation broth.
1.3 g (1300 mg) THC calculated load-in: 54 mg/L max.

### Infusing Distilled Spirits Method 1

This method can be used to infuse vodka or other distilled spirits with cannabis. Referring to FIG. 2, the steps up to and including step the fractional distillation step 106 are used. Components 108a and 110 at the outlet of the fractional still can then be blended into the distilled spirit so as to yield 10-25 mg THC per fluid ounce. By adjusting the quantity of components 110 (“cannabinoids strip”), the loading of THC can be adjusted, and then standardized afterward by adjustment with straight distilled spirit which has no cannabis infusion. The component 108b can be added “to taste”. For example, including more or less of the terpenes depending on the desired taste palate of the beverage. The infused spirit can be kept at 0° C. for a period of 5-15 days, and then filtered to remove any particulate matter.

### Infusing Distilled Spirits Method 2

This is an alternative method that can be used to infuse vodka or other distilled spirits with cannabis. The method is similar to the above method, except that the fractional distillation step is not used. Referring to FIG. 2 the steps up to and including step the chilling step 214 are used. After the chilling step 214, the liquid is separated to remove plant lipids/waxes and fats. The liquid is blended into the distilled spirit so as to yield 10-25 mg THC per fluid ounce. By adjusting the quantity of liquid from chilling step 214 to distilled spirits, the loading of THC is adjusted. Consistent and standardized products are made by adjustment with straight distilled spirit which has no cannabis infusion. The infused spirit can be kept at 0° C. for a period of 5-15 days, and then filtered to remove any particulate matter.

As used herein the term “comprising” or “comprises” is used in reference to compositions, methods, and respective component(s) thereof, that are essential to the claimed invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the claimed invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A process for the preparation of a terpene-reduced cannabinoid adjunct, the process comprising:
a. contacting a first plant matter having a first cannabinoid to terpene (C/T) ratio with steam, wherein after contacting with steam the first C/T ratio of the first plant matter is greater than a C/T ratio of the first plant matter prior to contacting with steam;
b. contacting the first plant matter with a solvent at a temperature below about 0° C. for at least 1 hour producing a first solvent fraction;
c. separating the first solvent fraction from the first plant matter;
d. fractionating the first solvent fraction producing a second solvent fraction, a terpene fraction and a solvent-free cannabinoid fraction, wherein the solvent-free cannabinoid fraction has a second C/T ratio that is greater than the first C/T ratio, wherein the second C/T ratio of the solvent-free cannabinoid fraction is at least 100; and
e. combining the solvent-free cannabinoid fraction with a soluble fiber thereby producing the terpene-reduced cannabinoid adjunct.

2. The process according to claim 1, further comprising:
c1. reducing the volume of the first solvent fraction by at least 30%, wherein after reducing the volume, the first C/T ratio is greater than a C/T ratio prior to reducing the volume;
c2. cooling the first solvent fraction at a temperature below about 0° C. for at least 1hour; and
c3. separating a lipid containing fraction from the first solvent fraction.

3. The process according to claim 2, wherein reducing the volume of the first solvent fraction (step c1) comprises reducing the volume by at least about 60%.

4. The process according to claim 2, wherein cooling the first solvent fraction (step c2) is at a temperature in a range of about −40 to 0° C. for less than about 24 hours.

5. The process according to claim 1, wherein prior to contacting the first plant matter with the solvent (step b), the first plant matter is subjected to drying.

6. The process according to claim 5, wherein drying comprises placing the first plant matter in a dry environment for at least one day, wherein the dry environment has a relative humidity less than about 90% and a temperature in a range of 15-40° C.

7. The process according to claim 6, wherein the first plant matter is placed in the dry environment for 1-7 days, and wherein the humidity is less than about 85% and the temperature is in a range of 19-35° C.

8. The process according to claim 1, wherein prior to contacting the first plant matter with the solvent (step b), the first plant matter is subjected to a first trim, wherein the first trim comprises removal of stems and leaves from the first plant matter.

9. The process according to claim 1, wherein prior to contacting the first plant matter with the solvent (step b), the first plant matter is subjected to a decarboxylation, wherein after the decarboxylation the first C/T ratio is greater than a C/T ratio of the first plant matter prior to decarboxylation.

10. The process according to claim 9, wherein the decarboxylation comprises heating the first plant matter at a temperature in a range of about 90-120° C. for at least 10 min.

11. The process according to claim 1, wherein prior to contacting the first plant matter with the solvent (step b), the first plant matter is subjected to fine trim comprising isolating a flower from other plant components and using only the flower as the first plant matter which is contacted with the solvent.

12. The process according to claim 1, wherein the solvent is ethanol, a hydrocarbon or supercritical CO2.

13. The process according to claim 12, wherein the solvent is ethanol having a concentration in a range of about 60%-90% abv.

14. The process according to claim 1, wherein contacting the first plant matter with a solvent (step b) is at a temperature in a range of about −40 to 0° C. for less than about 5 days producing the first solvent fraction.

15. The process according to claim 1, wherein fractionating the first solvent fraction (step d) comprises fractional distillation.

16. The process according to claim 15, wherein the fractional distillation comprises using a spin band column.

17. The process according to claim 15, wherein the fractional distillation is conducted under a vacuum in a range of 0.5 to 50 torr.

18. The process according to claim 1 wherein the first plant matter comprises a plant of the genus Cannabis.

19. The process according to claim 18, wherein the plant is Cannabis sativa, Cannabis indica, Cannabis ruderalis, or hydrides of one or more of these.

20. The process according to claim 1, wherein the soluble fiber (step e) comprises any one or more of xantham gum, guar gum, maltose, fructose, and maltotriose.

21. A terpene-reduced cannabinoid adjunct made according to claim 1, comprising at least one of tetrahydrocannabinoid (THC), cannabidiol (CBD), cannabichromene (CBC), canabinol (CBN), cannabielsoin (CBE), cannnabicyclol (CBL), cannabicitran (CBT), or isomers thereof.

22. An edible product comprising the terpene-reduced cannabinoid adjunct according to claim 21.

23. The edible product according to claim 22, wherein the edible product is a solid, semisolid or liquid.

24. The edible product according to claim 22, wherein the edible product is a fermented product.

25. The edible product according to claim 22, wherein the terpene-reduced cannabinoid adjunct is homogeneously distributed throughout the edible product.

26. A method of making an edible product comprising;
combining a terpene-reduced cannabinoid adjunct made according to claim 1 with water and at least one edible ingredient, thereby providing an edible combination.

27. The method according to claim 26, wherein combining comprises homogenizing the terpene-reduced cannabinoid adjunct throughout the edible combination.

28. The method according to claim 26, wherein the edible ingredient is selected from any one or more of a starch, a sweetener, a flavoring agent, a coloring agent, a protein, an amino acid, a vitamin, a mineral, a fruit, an ester, a nutritional supplement, a nutraceutical, a spice, fiber, a food grade oil, a food grade fat, or an alcohol.

29. The method according to claim 28, wherein the protein is a gel.

30. The method according to claim 28, wherein at least one edible ingredient is a sweetener.

31. The method according to claim 30, wherein the sweetener is a complex or simple sugar.

32. The method according to claim 31, wherein combining comprises further adding a yeast and a plant protein, and fermenting at least a portion of the sugar.

33. The method according to claim 32, wherein fermenting comprises adding the yeast at a rate of 1 million cells/ml per Degree Plato (° P) liquid and with an initial temperature in a range between 5 and 20° C.

34. The method according to claim 26, further comprising partitioning the edible product into containers.

35. The method according to claim 26, further comprising pasteurizing the edible product.

36. The method according to claim 1, wherein the second C/T ratio is at least 1000, or at least 10,000.
